SEQUENCE LISTING (42 sequences)

SEQ ID NO: 1 - protein, 252 aa, Influenza virus
MSLLTEVETYVLSIIPSGPLKAEIAQRLEDVFAGKNTDLEVLMEWLKTRPILSPLTKGILGFVFTLTVPSERGLQHRRFVQNALNGNGDPNNIDKAAKLYRKLKREVTFHGAKEISLSYSAGALASCMGLIYNRMGAVTTEVAFGLVCATCEQIADSQHRSHGQMVTTTNPLIRHENRMVLASTTAKAMEQMAGSSEQAAEAMEVASQARQMVQAMRTIGTHPSSSAGLKNDLLENLQAYQKRMGVQMQRFK

SEQ ID NO: 2 - protein, 121 aa, Influenza virus
MDPNTVSSFQDILLRMSKMQLESSSGDLNGMITQFESLKLYRDSLGEAVMRMGDLHSLQNRNEKWREQLGQKFEEIRWLIEEVRHKLKITENSFEQITFMQALHLLLEVEQEIRTFSFQLI

SEQ ID NO: 3 - protein, 498 aa, Influenza virus
MASQGTKRSYEQMETDGERQNATEIRASVGKMIGGIGRFYIQMCTELKLSDYEGRLIQNSLTIERMVLSAFDERRNKYLEEHPSAGKDPKKTGGPIYRRVNGKWMRELILYDKEEIRRIWRQANNGDDATAGLTHMMIWHSNLNDATYQRTRALVRTGMDPRMCSLMQGSTLPRRSGAAGAAVKGVGTMVMELVRMIKRGINDRNFWRGENGRKTRIAYERMCNILKGKFQTAAQKAMMDQVRESRNPGNAEFEDLTFLARSALILRGSVAHKSCLPACVYGPAVASGYDFEREGYSLVGIDPFRLLQNSQVYSLIRPNENPAHKSQLVWMACHSAAFEDLRVLSFIKGTKVLPRGKLSTRGVQIASNENMETMESSTLELRSRYWAIRTRSGGNTNQQRASAGQISIQPTFSVQRNLPFDRTTIMAAFNGNTEGRTSDMRTEIIRMMESARPEDVSFQGRGVFELSDEKAASPIVPSFDMSNEGSYFFGDNAEEYDN

SEQ ID NO: 4 - RNA, 68 nt, Artificial
  aptamer M1R9C1
gguuaccagccuucacugccugaccacucagaaucgagcgcauuggccggcaccacggucggucacac

SEQ ID NO: 5 - RNA, 68 nt, Artificial
  aptamer M1R9C6
gguuaccagccuucacugcccugaccauccugagggacgcauuggccgggcaccacggucggucacac

SEQ ID NO: 6 - RNA, 68 nt, Artificial
  aptamer M1R9C9
gguuaccagccuucacugccugaccauccuauuccggacgcauuggccggcaccacggucggucacac

SEQ ID NO: 7 - RNA, 68 nt, Artificial
  aptamer M1R9C20
gguuaccagccuucacugcccugaccaccuagauaggcgcauuggccgggcaccacggucggucacac

SEQ ID NO: 8 - RNA, 36 nt, Artificial
  aptamer M1R9C1 36 bases length
ugccugaccacucagaaucgagcgcauuggccggca

SEQ ID NO: 9 - RNA, 36 nt, Artificial
  aptamer M1R9C6 36 bases lenght
ugcccugaccauccugagggacgcauuggccgggca

SEQ ID NO: 10 - DNA, 36 bp, Artificial
  aptamer M1R9C1 ARN/DNA
ugccugaccacucagaatcgagcgcauuggccggca

SEQ ID NO: 11 - DNA, 36 bp, Artificial
  aptamer M1R9C6 ARN/DNA
ugcccugaccaucctgagggacgcauuggccgggca

SEQ ID NO: 12 - RNA, 6 nt, Artificial
  NS7
agaauc

SEQ ID NO: 13 - RNA, 4 nt, Artificial
  NS7
ugag

SEQ ID NO: 14 - RNA, 6 nt, Artificial
  NS7
uauucc

SEQ ID NO: 15 - RNA, 4 nt, Artificial
  NS7
agau

SEQ ID NO: 16 - DNA, 6 bp, Artificial
  NS7
agaatc

SEQ ID NO: 17 - DNA, 4 bp, Artificial
  NS7
tgag

SEQ ID NO: 18 - RNA, 3 nt, Artificial
  NS1
gcc

SEQ ID NO: 19 - RNA, 3 nt, Artificial
  NS2
ggc

SEQ ID NO: 20 - RNA, 4 nt, Artificial
  NS1
gccc

SEQ ID NO: 21 - RNA, 4 nt, Artificial
  NS2
gggc

SEQ ID NO: 22 - RNA, 3 nt, Artificial
  NS3
cca

SEQ ID NO: 23 - RNA, 3 nt, Artificial
  NS4
ugg

SEQ ID NO: 24 - RNA, 3 nt, Artificial
  NS5
cuc

SEQ ID NO: 25 - RNA, 3 nt, Artificial
  NS6
gag

SEQ ID NO: 26 - RNA, 3 nt, Artificial
  NS5
ucc

SEQ ID NO: 27 - RNA, 3 nt, Artificial
  NS6
ucc

SEQ ID NO: 28 - RNA, 3 nt, Artificial
  NS5
ccu

SEQ ID NO: 29 - RNA, 3 nt, Artificial
  NS6
agg

SEQ ID NO: 30 - DNA, 39 bp, Artificial
  ssDNA
gtgtgaccgaccgtggtgcngcagtgaaggctggtaacc

SEQ ID NO: 31 - DNA, 19 bp, Artificial
  primer p20
gtgtgaccgaccgtggtgc

SEQ ID NO: 32 - DNA, 36 bp, Artificial
  primer
taatacgactcactataggttaccagccttcactgc

SEQ ID NO: 33 - DNA, 10 bp, Artificial
  probe
tgccggccaa

SEQ ID NO: 34 - DNA, 10 bp, Artificial
  probe
tgcccggcca

SEQ ID NO: 35 - DNA, 26 bp, Artificial
  aptamer M1R9C1 26 bases-length
ugccugaccacucagaaucgagcgca

SEQ ID NO: 36 - DNA, 26 bp, Artificial
  M1R9C1 RNA/DNA aptamer 26 bases-length
ugccugaccacucagaatcgagcgca

SEQ ID NO: 37 - RNA, 26 nt, Artificial
  M1R9C6 aptamer 26 bases-length
ugcccugaccauccugagggacgcau

SEQ ID NO: 38 - DNA, 26 bp, Artificial
  M1R9C6 RNA/DNA aptamer 26 bases-length
ugcccugaccaucctgagggacgcau

SEQ ID NO: 39 - DNA, 36 bp, Artificial
  M1R9C1 DNA
tgcctgaccactcagaatcgagcgcattggccggca

SEQ ID NO: 40 - RNA, 31 nt, Artificial
  anti-VEGF
ggcgaaccgauggaauuuuuggacgcucgcc

SEQ ID NO: 41 - DNA, 34 bp, Artificial
  anti-PDGF
caggctacggcacgtagagcatcaccatgatcct

SEQ ID NO: 42 - RNA, 37 nt, Artificial
  MIR9C6 bulge less
ugcccuggccauccugagggacgcauuggccagggca

(19) United States
(12) Patent Application Publication
(10) Pub. No.: US 2012/0295811 A1
(43) Pub. Date: Nov. 22, 2012

(54) APTAMERS DIRECTED AGAINST THE MATRIX PROTEIN-1 OF TYPE A INFLUENZA VIRUSES AND USES THEREOF

(76) Inventors: Jean-Jacques Toulme, Bordeaux (FR); Eric Dausse, Bordeaux (FR); Francois Cornet, Liege (BE); Daniel Desmecht, Liege (BE)

(21) Appl. No.: 13/511,152

(22) PCT Filed: Nov. 23, 2010
(86) PCT No.: PCT/EP10/68062

§ 371 Date: Jul. 31, 2012

(30) Foreign Application Priority Data
Nov. 23, 2009 (EP) 09306126.5
Nov. 23, 2009 (EP) 09306130.7

(51) Int. Cl. C07H 21/00; C40B 30/04; C40B 40/06

(52) U.S. Cl. 506/9; 536/23.1; 506/16

(57) ABSTRACT

The present invention relates to nucleic acids that bind specifically to the matrix protein-1 of type A influenza viruses and uses thereof for detecting such viruses in a sample of interest. More particularly, the present invention relates to a nucleic acid that binds specifically to matrix protein-1 of type A influenza viruses characterized in that said nucleic acid comprises the following nucleotide sequence: 5′-N1-NS1-U-N3-A-NS3-NS5-NS7-NS6-CGCAU-NS4-C-N4-NS2-N2-3′ wherein: -N1 consists of a nucleotide -NS1 and NS2 consist of polynucleotides having 3 or 4 nucleotides in length, and NS1 and NS2 have complementary sequences; -N3 and N4 consists of a nucleotide, and N4 is complementary to N3; -NS3 and NS4 consist of polynucleotides having 3 nucleotides in length, and -NS3 and NS4 have complementary sequences -NS5 and NS6 consist of polynucleotides having 3 nucleotides in length, and -NS5 and NS6 have complementary sequences; -NS7 consists of a polynucleotide selected from the group consisting of AGAAUC (SEQ ID NO:12), UGAG (SEQ ID NO: 13), UAUUCC (SEQ ID NO:14), AGAU (SEQ ID NO:15), AGAATC (SEQ ID NO:16) or TGAG (SEQ ID NO:17) -N2 consists of a nucleotide that is complementary or not complementary to nucleotide N1.

### FIELD OF THE INVENTION

The present invention relates to nucleic acids that bind specifically to the matrix protein-1 of type A influenza viruses and uses thereof for detecting such viruses in a sample of interest.

### BACKGROUND OF THE INVENTION

Influenza is an orthomyxovirus with three genera, types A, B, and C. Types A and B are the most clinically significant since they causes acute respiratory infections that are highly contagious and afflict humans and animals with significant morbidity and mortality.

Type A viruses are principally classified into antigenic sub-types on the basis of two viral surface glycoproteins, hemagglutinin (HA) and neuraminidase (NA). There are currently 16 identified HA sub-types (designated H1 through H16) and 9 NA sub-types (N1 through N9) all of which can be found in wild aquatic birds. Of the 135 possible combinations of HA and NA, only four (H1N1, H1N2, H2N2, and H3N2) have widely circulated in the human population since the virus was first isolated in 1933. The matrix protein-1 is also a major structural component of influenza viruses located on the interior side of the viral envelope. It is also the most invariant antigen of type A influenza viruses.

Current public and scientific concern over the possible emergence of a pandemic strain of influenza requires earlier diagnosis of influenza viruses. Therefore there is an incentive for specific probes that will help physicians to detect influenza viruses in samples of interest.

For examples, antibodies directed against the matrix protein-1 have been developed as probes for detecting influenza viruses in clinical specimen (Bucher et al. (1991)).

Aptamers are a class of molecule that represents an alternative to antibodies in term of molecular recognition. Aptamers are oligonucleotide or oligopeptide sequences with the capacity to recognize virtually any class of target molecules with high affinity and specificity. Such ligands may be isolated through Systematic Evolution of Ligands by EXponential enrichment (SELEX) of a random sequence library, as described in Tuerk C. and Gold L., 1990. Accordingly, several aptamers directed against hemaglutinins of influenza viruses have been developed (see for example Gopinath S C, Kawasaki K, Kumar P K. Selection of RNA-aptamers against human influenza B virus. Nucleic Acids Symp Ser (Oxf). 2005; (49):85-6.).

### SUMMARY OF THE INVENTION

The present invention relates to nucleic acids that bind specifically to the matrix protein-1 of type A influenza viruses and uses thereof for detecting such viruses in a sample of interest.

More particularly, the present invention relates to a nucleic acid that binds specifically to matrix protein-1 of type A influenza viruses characterized in that said nucleic acid comprises the following nucleotide sequence:

5′-N1-NS1-U-N3-A-NS3-NS5-NS7-NS6-CGCAU-NS4-C-N4-
NS2-N2-3′

wherein:
N1 consists of a nucleotide
NS1 and NS2 consist of polynucleotides having 3 or 4 nucleotides in length, and NS1 and NS2 have complementary sequences;
N3 and N4 consists of a nucleotide, and N4 is complementary to N3;
NS3 and NS4 consist of polynucleotides having 3 nucleotides in length, and NS3 and NS4 have complementary sequences
NS5 and NS6 consist of polynucleotides having 3 nucleotides in length, and NS5 and NS6 have complementary sequences;
NS7 consists of a polynucleotide selected from the group consisting of AGAAUC (SEQ ID NO:12), UGAG (SEQ ID NO:13), UAUUCC (SEQ ID NO:14), AGAU (SEQ ID NO:15), AGAATC (SEQ ID NO:16) or TGAG (SEQ ID NO:17)
N2 consists of a nucleotide that is complementary or not complementary to nucleotide N1.

The present invention also relates to use of a nucleic acid of the invention for detecting or quantifying the matrix protein-1 in a sample of interest.

The present invention also relates to use of a nucleic acid of the invention for detecting and/or quantifying a Influenza virus of type A in a sample of interest.

The present invention also relates to a microarray comprising a solid support which carries at least one nucleic acid according to any of claims 1 to 10.

The present invention also relates to a kit comprising at least one nucleic acid of the invention.

### DETAILED DESCRIPTION OF THE INVENTION

The inventors have identified a small collection of structurally-related nucleic acids (aptamers) that all are able to bind to matrix protein-1 of Influenza virus with high affinity (see EXAMPLE 1). Furthermore the inventors demonstrate that the aptamers of the invention are very suitable for the manufacturing of aptamer microarrays so as to detect with high efficiency the matrix protein-1 even in a complex medium (see EXAMPLE 2). Accordingly the aptamers of the invention could be use in various diagnostic systems in the aim of influenza detection.

Thus, an object of the present invention consists of a nucleic acid that binds specifically to a matrix protein-1.

As used herein the term “matrix protein-1” refers to the matrix protein-1 of influenza viruses. Matrix protein-1 is a type-specific antigen with a common antigenicity shared among all type A influenza viruses, whether the original source of isolation is human, avian, swine, equine, or other animal species. An exemplary native amino acid sequence encoding for matrix protein-1 is represented by SEQ ID NO:1.

The matrix protein-1-specific nucleic acids of the invention may also be termed herein “matrix protein-1 aptamers”, since several of them have initially been selected by performing the SELEX™ method. The matrix protein-1-specific nucleic acids of the invention consist of nucleic acid ligands of matrix protein-1.

As intended herein, the nucleic acids above are “matrix protein-1 specific” because (i) they bind with a high affinity with matrix protein-1 and (ii) they do not bind, or alternatively they bind with low affinity, with other proteins.

As shown in the examples herein, the matrix protein-1-specific nucleic acids according to the invention bind to matrix protein-1 with a dissociation constant (KD) of less than 50 nM, and usually of less than 20 nM, while embodiments of these nucleic acids are endowed with a dissociation constant (KD) of less than 5 nM, and in some cases even less than 2 nM. The dissociation constant (KD) may also be termed the “(KD) affinity value” throughout the present specification.

The dissociation constant (KD) of a complex formed between matrix protein-1 proteins and a nucleic acid according to the invention may be determined by performing any one of the techniques that are well known from the one skilled in the art. Embodiments of the method for determining a dissociation constant (KD) is fully illustrated in the examples herein.

As shown in the examples herein, the specificity of the nucleic acids according to the invention for matrix protein-1 is illustrated by the absence of binding, or in some embodiments the very low binding, of these nucleic acids to non matrix-1 proteins, including to closely related non-matrix protein-1 proteins like nonstructural protein-2 (SEQ ID NO:2), or nucleoprotein (SEQ ID NO:3).

Generally, a matrix protein-1 aptamer according to the invention may be selected from the group consisting of DNA molecules and RNA molecules. In the examples herein, matrix protein-1 aptamers consisting of RNA or DNA molecules are described.

More particularly, the present invention relates to a nucleic acid that binds specifically to matrix protein-1 of type A influenza viruses characterized in that said nucleic acid comprises the following nucleotide sequence:

5′-N1-NS1-U-N3-A-NS3-NS5-NS7-NS6-CGCAU-NS4-C-N4-
NS2-N2-3′

wherein:
N1 consists of a nucleotide
NS1 and NS2 consist of polynucleotides having 3 or 4 nucleotides in length, and NS1 and NS2 have complementary sequences;
N3 and N4 consists of a nucleotide, and N4 is complementary to N3;
NS3 and NS4 consist of polynucleotides having 3 nucleotides in length, and NS3 and NS4 have complementary sequences
NS5 and NS6 consist of polynucleotides having 3 nucleotides in length, and NS5 and NS6 have complementary sequences;
NS7 consists of a polynucleotide selected from the group consisting of AGAAUC (SEQ ID NO:12), UGAG (SEQ ID NO:13), UAUUCC (SEQ ID NO:14), AGAU (SEQ ID NO:15), AGAATC (SEQ ID NO:16) or TGAG (SEQ ID NO:17)
N2 consists of a nucleotide that is complementary or not complementary to nucleotide N1.

As used herein, a “nucleotide” is selected from the group consisting of A, T, U, G or C, and any chemically modified form thereof.

In every matrix protein-1 aptamer according to the invention, the various “NS” sequences are included in a stem secondary structure, with a given first NS sequence being complementary to a given second NS sequence, excepting for NS7. Thus, when present in the nucleic acid sequence of a matrix protein-1 aptamer according to the invention, (i) NS1 and NS2 are complementary and form together a double-stranded stem secondary structure, as it is the case also for (ii) NS3 and NS4, and (iii) NS5 and NS6. The specific nucleic acid sequence of a given NSx (excepting for NS7) sequence is not essential, provided that the base pair complementarily between two given NSx sequences is ensured for forming the corresponding stem region of the matrix protein-1 aptamer under consideration.

In a particular embodiment, N1 is U and N2 is A.

In another particular embodiment, NS1 and NS2 consist of polynucleotides having 3 nucleotides in length. More particularly, NS1 is GCC (SEQ ID NO:18) and NS2 is GGC (SEQ ID NO:19).

In another particular embodiment, NS1 and NS2 consist of polynucleotides having 4 nucleotides in length. More particularly, NS1 is GCCC (SEQ ID NO:20) and NS2 is GGGC (SEQ ID NO:21).

In another particular embodiment, N3 is G and N4 is C.

In another particular embodiment, NS3 is CCA (SEQ ID NO:22) and NS4 is UGG (SEQ ID NO:23).

In another particular embodiment, NS5 is CUC (SEQ ID NO:24) and NS6 is GAG (SEQ ID NO:25).

In another particular embodiment, NS5 is UCC (SEQ ID NO:26) and NS6 is GGA (SEQ ID NO:27).

In another particular embodiment, NS5 is CCU (SEQ ID NO:28) and NS6 is AGG (SEQ ID NO:29).

In another particular embodiment, the nucleic acid of the invention comprises or consists of a nucleic acid sequence selected from the group consisting of SEQ ID NO:8 (M1R9C1 36 bases length), SEQ ID NO:9 (M1R9C6 36 bases length), SEQ ID NO:10 (M1R9C1 RNA/DNA 36 bases length) and SEQ ID NO:11 (M1R9C6 RNA/DNA 36 bases length).

In certain other embodiments of a matrix protein-1 aptamer according to the invention, the nucleic acid sequence of such a matrix protein-1 aptamer comprises a nucleic acid sequence as above described, and thus also comprises either (i) one additional nucleic acid sequence located at the 5′-end or at the 3′-end of the said aptamer or (ii) one additional nucleic acid sequence located at each of both the 5′-end and the 3′-end of the said aptamer. These additional nucleic acid sequences may have from 1 to 30 nucleotides in length, irrespective of the identity of the added sequence(s), without significantly altering the binding properties of the resulting aptamer to matrix protein-1. Thus, these additional nucleic acid sequences may have 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 nucleotides in length, while maintaining binding properties similar to the binding properties of the corresponding matrix protein-1 aptamer without the additional sequence(s), i.e. a (KD) dissociation constant which is at most distinct of one order of magnitude, as compared with the corresponding matrix protein-1 aptamer without the additional sequence(s).

As shown in the examples herein, illustrative matrix protein-1 aptamers as set forth in SEQ ID NO:4 and SEQ ID NO:5 comprise 19-mer additional sequences located both at the 5′-end and at the 3′-end of specific embodiments of the nucleic acid sequences as set forth in SEQ ID NO:8 and SEQ ID:9 respectively, while having binding properties which are similar with, if not identical to, the binding properties of the corresponding matrix protein-1 aptamers wherein these additional sequences are absent. The additional sequences may form secondary structure(s) of internal loop(s), stem(s), or both.

According to another particular embodiment, the nucleic acid of the invention comprises or consists of a nucleic acid sequence selected from the group consisting of SEQ ID NO:4 (M1R9C1) and SEQ ID NO:5 (M1R9C6).

Nucleic acids of the invention may be produced by any technique known in the art, such as, without limitation, any chemical, biological, genetic or enzymatic technique, either alone or in combination. Knowing the nucleic acid sequence of the desired sequence, one skilled in the art can readily produce said aptamers, by standard techniques for production of polynucleotides. For instance, they can be synthesized using well-known solid phase method, preferably using a commercially available polynucleotide synthesis apparatus.

In preferred embodiments, any one of the matrix protein-1 aptamers according to the present invention may be chemically modified, so as to increase its chemical stability both in vitro and in vivo, and notably so as to decrease its degradation by cellular enzymes, typically its degradation by exonucleases and endonucleases. Chemically modified matrix protein-1 aptamers are particularly suitable for their use in vivo, either as such or combined with active compounds like protease inhibitors for medical purposes.

One potential problem encountered in the use of nucleic acids is that oligonucleotides in their phosphodiester form may be quickly degraded in body fluids by intracellular and extracellular enzymes such as endonucleases and exonucleases before the desired effect is manifest. The SELEX™ method thus encompasses the identification of high-affinity nucleic acid ligands containing modified nucleotides conferring improved characteristics on the ligand, such as improved in vivo stability or improved delivery characteristics. Examples of such modifications include chemical substitutions at the sugar and/or phosphate and/or base positions. SELEX™-identified nucleic acid ligands containing modified nucleotides are described, e.g., in U.S. Pat. No. 5,660,985, which describes oligonucleotides containing nucleotide derivatives chemically modified at the 2′ position of ribose, 5 position of pyrimidines, and 8 position of purines, U.S. Pat. No. 5,756,703 which describes oligonucleotides containing various 2′-modified pyrimidines, and U.S. Pat. No. 5,580,737 which describes highly specific nucleic acid ligands containing one or more nucleotides modified with 2′-amino (2′-NH.sub.2), 2′-fluoro (2′-F), and/or 2′-OMe substituents. Techniques 2′-chemical modification of nucleic acids are also described in the U.S. patent applications N° US 2005/0037394 and N° US 2006/0264369.

Modifications of the nucleic acid ligands contemplated in this invention include, but are not limited to, those which provide other chemical groups that incorporate additional charge, polarizability, hydrophobicity, hydrogen bonding, electrostatic interaction, and fluxionality to the nucleic acid ligand bases or to the nucleic acid ligand as a whole. Modifications to generate oligonucleotide populations which are resistant to nucleases can also include one or more substitute internucleotide linkages, altered sugars, altered bases, or combinations thereof. Such modifications include, but are not limited to, 2′-position sugar modifications, 5-position pyrimidine modifications, 8-position purine modifications, modifications at exocyclic amines, substitution of 4-thiouridine, substitution of 5-bromo or 5-iodo-uracil, backbone modifications, phosphorothioate or alkyl phosphate modifications, methylations, and unusual base-pairing combinations such as the isobases isocytidine and isoguanidine. Modifications can also include 3′ and 5′ modifications such as capping.

In one embodiment, oligonucleotides are provided in which the P(O)O group is replaced by P(O)S (“thioate”), P(S)S (“dithioate”), P(O)NR.sub.2 (“amidate”), P(O)R, P(O)OR′, CO or CH.sub.2 (“formacetal”) or 3′-amine (—NH—CH.sub.2-CH.sub.2-), wherein each R or R′ is independently H or substituted or unsubstituted alkyl. Linkage groups can be attached to adjacent nucleotides through an —O—, —N—, or —S— linkage. Not all linkages in the oligonucleotide are required to be identical. As used herein, the term phosphorothioate encompasses one or more non-bridging oxygen atoms in a phosphodiester bond replaced by one or more sulfur atoms

In further embodiments, the oligonucleotides comprise modified sugar groups, for example, one or more of the hydroxyl groups is replaced with halogen, aliphatic groups, or functionalized as ethers or amines. In one embodiment, the 2′-position of the furanose residue is substituted by any of an O-methyl, O-alkyl, O-allyl, S-alkyl, S-allyl, or halo group. Methods of synthesis of 2′-modified sugars are described, e.g., in Sproat, et al., Nucl. Acid Res. 19:733-738 (1991); Cotten, et al., Nucl. Acid Res. 19:2629-2635 (1991); and Hobbs, et al, Biochemistry 12:5138-5145 (1973). Other modifications are known to one of ordinary skill in the art. Such modifications may be pre-SELEX™ process modifications or post-SELEX™πprocess modifications (modification of previously identified unmodified ligands) or may be made by incorporation into the SELEX™ process.

Pre-SELEX™ process modifications or those made by incorporation into the SELEX™ process yield nucleic acid ligands with both specificity for their SELEX™ target and improved stability, e.g., in vivo stability. SELEX™ process modifications made to nucleic acid ligands may result in improved stability, e.g., in vivo stability without adversely affecting the binding capacity of the nucleic acid ligand.

The SELEX™ method encompasses combining selected oligonucleotides with other selected oligonucleotides and non-oligonucleotide functional units as described in U.S. Pat. No. 5,637,459 and U.S. Pat. No. 5,683,867. SELEX™ method further encompasses combining selected nucleic acid ligands with lipophilic or non-immunogenic high molecular weight compounds in a diagnostic or therapeutic complex, as described, e.g., in U.S. Pat. No. 6,011,020, U.S. Pat. No. 6,051,698, and PCT Publication No. WO 98/18480. These patents and applications teach the combination of a broad array of shapes and other properties, with the efficient amplification and replication properties of oligonucleotides, and with the desirable properties of other molecules.

Thus, in certain embodiments of the matrix protein-1 aptamers according to the invention, the said matrix protein-1 aptamers are protected against hydrolysis by nucleases by chemical modification.

In certain preferred embodiments, the said chemical modification of a matrix protein-1 aptamer consists of incorporation of 2′-Fluoro groups into the nucleotides included in the matrix protein-1 aptamer nucleic acid.

In certain embodiments of the matrix protein-1 aptamers according to the invention, said matrix protein-1 aptamers are useful as means for detecting and/or quantifying the matrix protein-1 protein (or a type A influenza virus) in a sample of interest. For these detection purposes, the use of matrix protein-1 aptamers which are labeled with a detectable molecule may be useful, so as to easily detect and/or quantify the complexes formed between (i) the matrix protein-1 protein molecules present in the sample to be tested and (ii) the matrix protein-1 aptamer molecules.

Diagnostic agents need only be able to allow the user to identify the presence of a given target at a particular locale or concentration. Simply the ability to form binding pairs with the target may be sufficient to trigger a positive signal for diagnostic purposes. Those skilled in the art would be able to adapt any matrix protein-1 aptamer by procedures known in the art to incorporate a marker in order to track the presence of the said matrix protein-1 aptamer, either under the form of a free unbound molecule or in contrast as a molecule bound to the target matrix protein-1 protein.

The matrix protein-1 aptamers according to the invention may be labelled with a detectable molecule, such as, for example, a radioisotope, a fluorescent compound, a bioluminescent compound, a chemiluminescent compound, a metal chelator or an enzyme.

Thus, a matrix protein-1 aptamer according to the invention may be labelled by incorporating a label which is detectable by a method selected form the group comprising spectroscopic, photochemical, fluorescence, biochemical, immunochemical or chemical means. Useful detectable molecules include radioactive substances (32P, 35S, 3H, 125I), fluorescent dyes (5-bromodesoxyuridin, fluorescein, acteylaminofluorene, digoxygenin) or biotin.

The matrix protein-1 aptamers according to the invention may be labelled at their 3′-end or 5′-end nucleotides without significantly altering their binding properties to matrix protein-1.

Another object of the invention consists of a method for detecting and/or quantifying the matrix protein-1 in a sample of interest.

Thus, the present invention also relates to a method for detecting the presence of matrix protein-1 proteins in a sample comprising the steps of:

a) providing a sample to be tested;

b) bringing into contact said sample with one or more matrix protein-1 aptamers that are described throughout the present specification;

c) detecting the complexes eventually formed between the matrix protein-1 proteins and the said nucleic acids.

The present invention also deals with a method for quantifying the presence of matrix protein-1 proteins in a sample comprising the steps of:

a) providing a sample to be tested;

b) bringing into contact said sample with one or more matrix protein-1 aptamers that are described throughout the present specification;

c) quantifying the complexes eventually formed between the matrix protein-1 proteins and the said nucleic acids.

Another object of the invention consists of method for detecting and/or quantifying a Influenza virus of type A in a sample of interest.

Thus, the present invention also relates to a method for detecting the presence of a type A influenza virus in a sample comprising the steps of:

a) providing a sample to be tested;

b) bringing into contact the said sample with one or more matrix protein-1 aptamers that are described throughout the present specification;

c) detecting the complexes eventually formed between the matrix protein-1 proteins and the said nucleic acids wherein the presence of such complexes is indicative of the presence of a type A influenza virus in said sample.

The present invention also relates to a method for quantifying the presence of a type A influenza virus in a sample comprising the steps of:

a) providing a sample to be tested;

b) bringing into contact the said sample with one or more matrix protein-1 aptamers that are described throughout the present specification;

c) quantifying the complexes eventually formed between the matrix protein-1 proteins and the said nucleic acids wherein the presence of such complexes is indicative of the presence of a type A influenza virus in said sample.

A “sample of interest” according to the invention encompasses a variety of sample types obtained from a subject and can be used in a diagnostic assay. Samples herein may be any type of sample, such as an individual's sample, or a culture sample containing or suspected of containing a type A influenza virus, including but not limited to laboratory cultures, nasopharangeal washes, expectorate, respiratory tract swabs, throat swabs, tracheal aspirates, bronchoalveolar lavage, mucus and saliva. In one embodiment, a sample contemplated by the invention may include any mammal known to harbor influenza, including but not limited to humans, birds, horses, dogs, cats and swine.

Detection or quantification methods of a target molecule using aptamer ligands that are specifically directed against the said target molecule are well known in the art and may be thus be performed by the one skilled in the art.

Notably, the one skilled in the art may perform any one of the detection/quantification methods that are disclosed in the examples herein, including detection or quantification of the complexes formed between (i) a matrix protein-1 aptamer previously immobilized on a microarray and (ii) matrix protein-1.

Generally, the detection and/or quantification methods of the invention, using a matrix protein-1 aptamer according to the invention, can be conducted in a variety of ways.

For example, one method to conduct such an assay would involve anchoring the matrix protein-1 aptamer onto a microarray and detecting matrix protein-1/matrix protein-1 aptamer complexes anchored on the microarray at the end of the reaction. In a particular embodiment, the matrix protein-1 aptamer is therefore anchored to a microarray and a sample from a subject can be allowed to react as an unanchored component of the assay.

There are many established methods for anchoring assay components to a microarray. These include, without limitation, matrix protein-1 aptamer molecules which are immobilized through conjugation of biotin and streptavidin. Such biotinylated assay components can be prepared from biotin-NHS (N-hydroxy-succinimide) using techniques known in the art (e.g., biotinylation kit, Pierce Chemicals, Rockford, Ill.), and immobilized in the wells of streptavidin-coated 96 well plates (Pierce Chemical). In certain embodiments, the surfaces with immobilized assay components can be prepared in advance and stored.

Other suitable carriers or microarray supports for such assays include any material capable of binding the matrix protein-1 aptamers. Well-known supports or carriers include, but are not limited to, glass, polystyrene, nylon, polypropylene, nylon, polyethylene, dextran, amylases, natural and modified celluloses, polyacrylamides, gabbros, and magnetite.

A further object of the invention consists of a microarray that allows performing the methods of the invention comprising a solid support which carries at least one nucleic acid of the invention.

In order to conduct assays with the above mentioned approaches, the non-immobilized component is added to the microarray upon which the second component is anchored. After the reaction is complete, uncomplexed components may be removed (e.g., by washing) under conditions such that any complexes formed will remain immobilized upon the microarray.

The detection of matrix protein-1/matrix protein-1 aptamer complexes anchored to the microarray can be accomplished in a number of methods outlined herein.

In a preferred embodiment, the matrix protein-1, when it is the unanchored assay component, can be labelled for the purpose of detection and readout of the assay, either directly or indirectly, with detectable labels discussed herein and which are well-known to one skilled in the art.

It is also possible to directly detect matrix protein-1/matrix protein-1 aptamer complex formation without further manipulation or labelling of either component (matrix protein-1 or matrix protein-1 aptamer), for example by utilizing the technique of fluorescence energy transfer (see, for example, Lakowicz et al., U.S. Pat. No. 5,631,169; Stavrianopoulos, et al., U.S. Pat. No. 4,868,103). A fluorophore label on the first, ‘donor’ molecule is selected such that, upon excitation with incident light of appropriate wavelength, its emitted fluorescent energy will be absorbed by a fluorescent label on a second ‘acceptor’ molecule, which in turn is able to fluoresce due to the absorbed energy. Alternately, the ‘donor’ protein molecule may simply utilize the natural fluorescent energy of tryptophan residues. Labels are chosen that emit different wavelengths of light, such that the ‘acceptor’ molecule label may be differentiated from that of the ‘donor’. Since the efficiency of energy transfer between the labels is related to the distance separating the molecules, spatial relationships between the molecules can be assessed. In a situation in which binding occurs between the molecules, the fluorescent emission of the ‘acceptor’ molecule label in the assay should be maximal. A FRET binding event can be conveniently measured through standard fluorometric detection means well known in the art (e.g., using a fluorimeter).

In another embodiment, determination of the ability of a probe to recognize a marker can be accomplished without labeling either assay component (probe or marker) by utilizing a technology such as real-time Biomolecular Interaction Analysis (BIA) (see, e.g., Sjolander, S. and Urbaniczky, C., 1991, Anal. Chem. 63:2338-2345 and Szabo et al., 1995, Curr. Opin. Struct. Biol. 5:699-705). As used herein, “BIA” or “surface plasmon resonance” is a technology for studying biospecific interactions in real time, without labeling any of the interactants (e.g., BIAcore). Changes in the mass at the binding surface (indicative of a binding event) result in alterations of the refractive index of light near the surface (the optical phenomenon of surface plasmon resonance (SPR)), resulting in a detectable signal which can be used as an indication of real-time reactions between biological molecules.

Alternatively, in another embodiment, analogous diagnostic assays can be conducted with matrix protein-1 and matrix protein-1 aptamer as solutes in a liquid phase. In such an assay, the complexed matrix protein-1 and matrix protein-1 aptamer are separated from uncomplexed components by any of a number of standard techniques, including but not limited to: differential centrifugation, chromatography, electrophoresis and immunoprecipitation. In differential centrifugation, matrix protein-1/matrix protein-1 aptamer complexes may be separated from uncomplexed assay components through a series of centrifugal steps, due to the different sedimentation equilibria of complexes based on their different sizes and densities (see, for example, Rivas, G., and Minton, A. P., 1993, Trends Biochem Sci. 18 (8):284-7). Standard chromatographic techniques may also be utilized to separate complexed molecules from uncomplexed ones. For example, gel filtration chromatography separates molecules based on size, and through the utilization of an appropriate gel filtration resin in a column format, for example, the relatively larger complex may be separated from the relatively smaller uncomplexed components. Similarly, the relatively different charge properties of the marker/probe complex as compared to the uncomplexed components may be exploited to differentiate the complex from uncomplexed components, for example through the utilization of ion-exchange chromatography resins. Such resins and chromatographic techniques are well known to one skilled in the art (see, e.g., Heegaard, N. H., 1998, J. Mol. Recognit. Winter 11(1-6):141-8; Hage, D. S., and Tweed, S. A. J Chromatogr B Biomed Sci Appl 1997 Oct. 10; 699(1-2):499-525). Gel electrophoresis may also be employed to separate complexed assay components from unbound components (see, e.g., Ausubel et al., ed., Current Protocols in Molecular Biology, John Wiley & Sons, New York, 1987-1999). In this technique, protein/nucleic acid complexes are separated based on size or charge, for example. In order to maintain the binding interaction during the electrophoretic process, non-denaturing gel matrix materials and conditions in the absence of reducing agent are typically preferred. SELDI-TOF technique may also be employed on matrix or beads coupled with active surface, or not, or antibody coated surface, or beads.

In another preferred embodiment of the detection or quantification methods above, these include the use of an optical biosensor such as described by Edwards and Leatherbarrow (Edwards and Leatherbarrow, 1997, Analytical Biochemistry, 246: 1-6) or also by Szabo et al. (Szabo et al., 1995, Curr. Opinion Struct. Biol., 5 (5): 699-705). This technique allows the detection of interactions between molecule in real time, without the need of labelled molecules. This technique is based on the surface plasmon resonance (SPR) phenomenon. Briefly, matrix protein-1 aptamer molecules are attached to a surface (such as a carboxymethyl dextran matrix). Then, the sample to be tested is incubated with the previously immobilised matrix protein-1 aptamers. Then, the binding, including the binding level, or the absence of binding between the matrix protein-1 aptamers and the matrix protein-1 protein molecules eventually present in the tested sample is detected. For this purpose, a light beam is directed towards the side of the surface area of the substrate that does not contain the sample to be tested and is reflected by said surface. The SPR phenomenon causes a decrease in the intensity of the reflected light with a specific combination of angle and wavelength. The binding of the matrix protein-1 aptamers and the matrix protein-1 molecules causes a change in the refraction index on the substrate surface, which change is detected as a change in the SPR signal. This technique is fully illustrated in the examples herein.

Other embodiments of the detection or quantification of matrix protein-1 molecules in a sample include the use of matrix protein-1 aptamers immobilized within a sol-gel matrix (as disclosed in the U.S. Patent application No. US 2006/0068407) or the use of matrix protein-1 aptamer-nanoparticle conjugates (as described in the U.S. Patent application No. US 2006/0014172).

This invention further pertain to kits for detecting or for performing the methods of the invention, wherein the said kits comprise one or more matrix protein-1 aptamers according to the invention, and optionally one more reagents that are necessary for performing a detection or a quantification methods as described herein.

The invention will be further illustrated by the following figures and examples. However, these examples and figures should not be interpreted in any way as limiting the scope of the present invention.

### FIGURES

FIG. 1: Evaluation of the RNA pool evolution. SPR analysis was performed on CM5 biochip after matrix protein-1 immobilization by amino groups. Fresh RNA pools came from selection were injected at 2 μM into the flow cell at flow rate of 10 μl/min during 10 minutes.

FIG. 2: Specificity study of the RNA pool for the target protein. SPR analyses were performed on immobilized proteins by amino groups on CM5. Fresh RNA pools (2 μM) were injected at the 10 μl/minute flow rate (A et B). The comparison of specificity between matrix protein-1 and nucleoprotein was realized with the RNA library (blank) and 9th round RNA pool (C).

FIG. 3: Predicted secondary structures of short aptamers: Prediction of structures were performed with mfold v3.2 Software (Zuker, 2003). http://frontend.bioinfo.rpi.edu/applications/mfold/cgi-bin/ma-form1.cgi.

FIG. 4: Evaluation of binding capacities of short aptamers. Comparative evaluation of 68 bases-length and 36 bases-length aptamers. Target protein was immobilized on CM5 biochip by amino groups. SPR analyses were performed at 20 μl/minute flow rate (22° C.) for injections of 100 μl at 2 μM.

FIG. 5: Specificity study of the short aptamers for target protein. SPR analyses were done on immobilized proteins by amino groups. Aptamers were injected at 2 μM (22° C., 20 μl/min).

FIG. 6: Quality control of aptamers immobilization on array. Quality control was performed with specific cy-3 labeled probes (10 nM) under 550 nm at 360 PMT. Oligonucleotides were arrayed on E slides in three replicate spots and immobilized by 5′-amino group. 1. spotting buffer; 2. oligo C1; 3. oligo C6; 4. tRNA; 5. M1R9C1 DNA; 6. anti-VEGF; 7. Anti-PDGF; 8. M1R9C1 bulgeless.

FIG. 7: Detection of matrix protein-1 by aptamer microarrays. Detection was performed with cy-5 labeled matrix protein-1 at 550 PMT. Samples were diluted 50× (right) or 250× (left) in the hybridization buffer from the stock solution (100 μg/ml) and dropped on low-density microarray (Top) and high-density microarray (Bottom).

FIG. 8: Detection of matrix protein-1 in complex medium. Detection was performed with cy-3 labeled matrix protein-1 and cy-5 labeled total proteins of Vero cells. Matrix protein-1 (100 μg/ml) was mixed in total protein Vero cells (0.5 mg/ml) at a ratio 1:1 and diluted 50× in the hybridization buffer. Fluorescence image were obtained on low-density microarray (left) and high-density microarray (right) under 750 PMT. Slides were successively scanned at 550 nm and 655 nm.

### EXAMPLE 1

### Characterization of the Aptamers

Materials and Methods:

Production and purification of target—The production of matrix protein-1 of Influenza A virus was performed with pET 14b-M1 expression vector while the productions of nucleoprotein and nonstructural protein-2 were respectively performed with pET 28a-NP and pET 16b-NS2. E. Coli (BL21/DE3) pLys cells were transformed and selected under chloramphenicol (17 μg/ml) and ampicillin (25 μg/ml) on agarose plate. The selection of E. Coli (BL21/DE3) pLys pET 28a-NP was done on kanamycin (50 μg/ml). Biomass was produced (37° C.; 200 rpm) in LB broth completed with chloramphenicol (17 μg/ml) and ampicillin (25 μg/ml) or kanamycin (50 μg/ml) to seed larger volumes of fresh medium (4×500 ml). At 0.6 O.D, protein expression was induced by addition of isopropylthio-β-D-galactosidase (0.8 mM). Production was performed during 16 h at 22° C. for M1, at 30° C. for NP and 37° C. for NS2. After centrifugation (10,000 g; 10 min; 4° C.), the pellet was suspended in 50 ml of icecold buffer (HEPES 50 mM pH 7.5; lysozyme 10 μg/ml from Sigma) plus four pills of antiprotease cocktail complete EDTA-free™ (Roche). Cells were lysed under high pressure (28 psi). The preparation was clarified by Benzonase treatment (10 minutes, 5 U/ml Merck) and was centrifuged (10,000 g; 10 min; 4° C.). The supernatant was maintained at 4° C. and equilibrated with NaCl (0.3 M) and imidazole (10 mM).

Purification was performed on Co²⁺ chelating resins according to the manufacturer (Sigma). Resin was equilibrated in buffer composed of HEPES (50 mM) pH 7.5, NaCl (0.3 M) and imidazole (10 mM). Elution of protein was performed with imidazole (200 mM). The degree of purity was estimated on 10% SDS-PAGE and on Western blot. Proteins were dialysed against phosphate buffer (Na₂PO₄ 10 mM pH 7.5; NaCl 137 mM; KCl 2.7 mM) and quantified using BradFord method.

Library—The library was designed as previously described (Dausse, E. et al [2005], Methods Mol Biol 288: 391-410). The library contained a central domain consisting of a randomized sequence (N30) flanked by known 5′ and 3′ arms. The ssDNA 5′-GTGTGACCGACCGTGGTGC-N30-GCAGTGAAGGCTGGTAACC-3′ (SEQ ID NO:30) (was amplified with Ampli Taq Gold (Applied Biosystems; #4311820) using 2 μM of each primer (P20: 5′-GTGTGACCGACCGTGGTGC-3′ (SEQ ID NO:31); 3′SL: 5′-TAATACGACTCACTATAGGTTACCAGCC TTCACTGC-3′ (SEQ ID NO:32)). The dsDNA was purified with phenol/chloroform—isoamylic alcohol and precipitated in the presence of sodium acetate (3 M pH 5.3). The RNA library was obtained after transcription for 2 hours at 37° C. using Ampliscribe T7 high yield transcription kit (TEBU; #AS3107). Two μl of RNase free DNase were added for 15 min. RNA candidates were purified by electrophoresis on denaturing 20% polyacrylamide, 7 M urea gels (15 watts/gel).

In vitro selection of matrix protein-1 binding aptamer—Selection was performed in SELEX buffer (Na₂PO₄ 10 mM pH 7.5; NaCl 137 mM; KCl 2.7 mM; (CH₃COO)₂ Mg 1 mM) at 22° C. during 45 min in microtubes. The first round of selection was carried out with 1000 pmol of the original library at RNA to protein 50:1 molar ratio. During selection the concentration of target was decreased keeping the molar ratio at 25:1 from the 4th round to the 9th. Before selection, RNA libraries were heated at 65° C. for 3 min, then ice cooled for 1 min, and finally kept at room temperature for 5 min. After each round of selection, unspecific RNAs were separated firstly by incubation with nitrocellulose membrane pieces of 1 square mm and secondly by incubation in the presence of 13 pmol of His tag SNEV (SeNescence EVasion factor) protein. After 45 min of incubation at room temperature, the matrix protein-1—RNA complexes were rescued by filtration through alkali-treated nitrocellulose membranes (Millipore; #HAWP02500) and washed twice with 1 ml of SELEX buffer. RNAs were recovered after denaturation with 7 M urea and Tris buffered phenol-chloroform pH 7.9. The recovered RNAs were reverse-transcribed for 50 min at 50° C. in 20 μl of reaction mixture containing primer P20 (2 μM) and using 240 units of M-MLV reverse transcriptase RNase H-point mutant kit (Promega; #M368A). The cDNA produced was amplified by PCR, transcribed and used for the next round of selection.

Cloning and sequencing—After nine rounds of selection against matrix protein-1, selected sequences were cloned using TOPO TA cloning kit (Invitrogen; #K460001) and sequenced with the BigDye terminator v1.1 cycle sequencing kit (Applied Biosystems; #4336697) according to the manufacturer's instructions. Sequences were analyzed and secondary structures were predicted with mfold 3.2 software (Zuker, M. (2003). Nucleic Acids Res 31 (13): 3406-15).

Analyses by Surface Plasmon Resonance (SPR)—Evaluations by SPR were carried out with Biacore 3000 equipment using a CM5 biochip at 22° C. Biochip was equilibrated with HBS pH 7.4 and activated with 35 μl of a 1:1 mixture of NHS (50 mM)/EDC (200 mM). For immobilization, proteins were mixed 1:1 in CH₃COONa (10 mM) pH 5 and injected at a flow rate of 5 μl/min. The biochip was saturated with 35 μl of ethanolamine (1 M) pH 8.5. To compare the binding abilities of RNA pools from different selection cycles, fresh transcription products were prepared. RNAs were mixed in SELEX buffer at 2 μM and folded (65° C. 5 min; 4° C. 1 min; room temperature 5 min). Evaluations were carried out at flow rate of 10 μl/min at 22° C. At the end of the injection, protein targets were regenerated with three pulses of 5 μl of NaOH (3 mM). Then, the integrated fluidic cartridge, needle and target were washed with the SELEX buffer.

The determination of impact of modifications of the sequences of selected aptamers was performed at the concentration of 2 μM in SELEX buffer at flow rate of 20 μl/min. In the same way, the affinity constants of aptamers were performed using a range of concentrations (0.2 to 10 μM) in SELEX buffer at flow rate of 20 μl/min. The sensorgrams were fitted to a kinetic titration 1:1 interaction model and analysed with the BIAeval software 2.2.4.

Results:

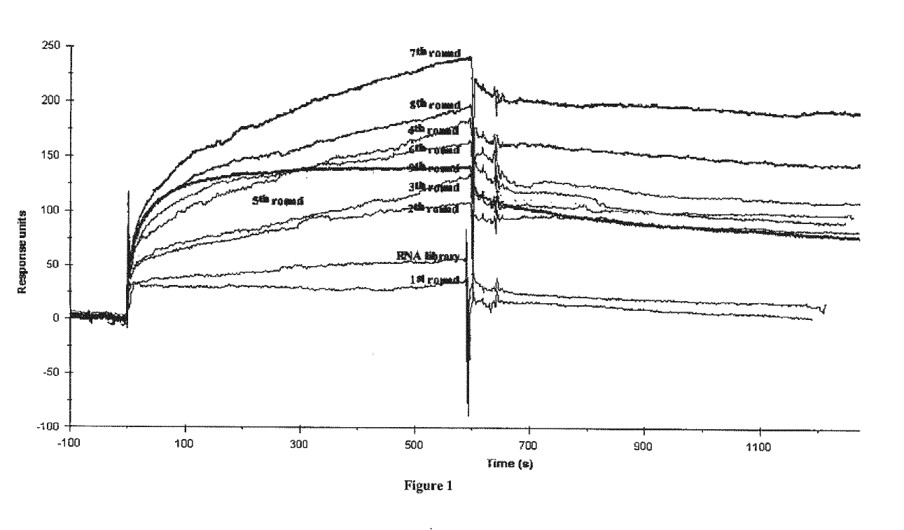

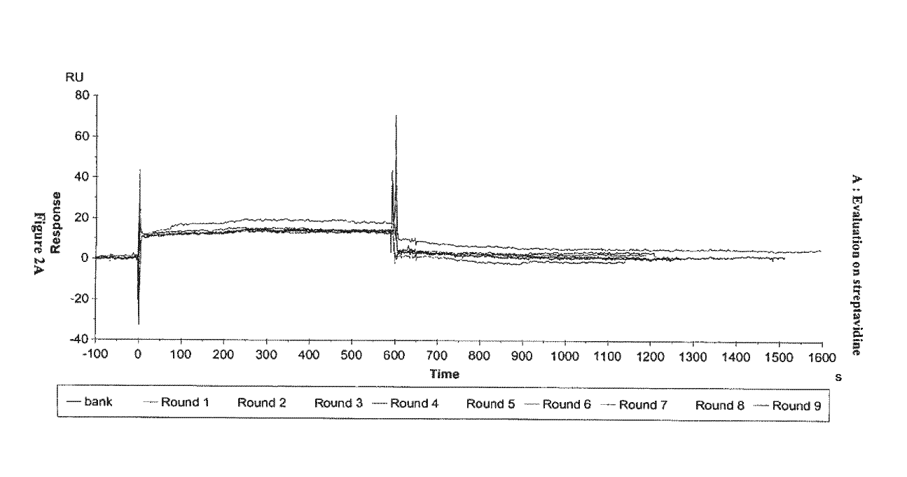

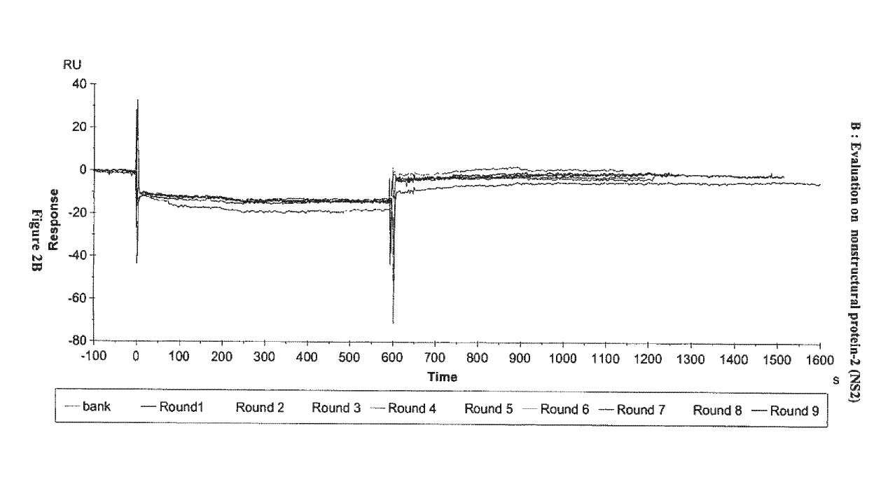

Characterization of aptamers selected—After nine rounds of selection, the evolution of RNA populations was analyzed by SPR. Selected RNAs of each cycle were injected on CM5 biochip coated with target proteins. Round after round, a gradual increase of the binding capacities between RNAs selected and matrix protein-1 (SEQ ID NO:1) was observed (FIG. 1). Concomitantly, RNA populations showed a low and stable affinity for streptavidin and for two nonpertinent viral proteins, nonstructural protein-2 (SEQ ID NO:2) and nucleoprotein (SEQ ID NO:3) (FIG. 2).

After reverse transcription, aptamers selected at the 9^{th} round were cloned. Sequence analysis revealed that the population of RNAs was composed mainly of the aptamer M1R9C1 (87.5%) (SEQ ID NO:4). The rest of the population was composed of three different aptamers showing some differences located in the randomized sequence (yellow) (SEQ ID NO:5, 6 and 7).

Individual study by SPR showed slight differences of binding capacities between the four selected aptamers. Kinetic components of the most efficient aptamers were determined using increasing concentrations of M1R9C1 and M1R9C6 from 0.1 to 10 μM (Table 2; 68 bases-length). Sensorgrams were fitted to kinetic titration 1:1 interaction model. Association (ka) and dissociation (kb) of M1R9C1 aptamer to matrix protein-1 were estimated around to 8×10³ (M⁻¹s⁻¹) and 2×10⁻³ (s⁻¹) respectively. The equilibrium dissociation constant (KD) for M1R9C1-M1 complexes was around 4×10⁻⁷ M.

The same kind of results were obtained to M1R9C6 aptamer for which association (ka) and dissociation constants (kb) were around of 4×10³ (M⁻¹s⁻¹) and 1×10⁻³ (s⁻¹), respectively. The equilibrium dissociation constant (KD) for M1R9C1-M1 complexes was around 3×10⁻⁷ M.

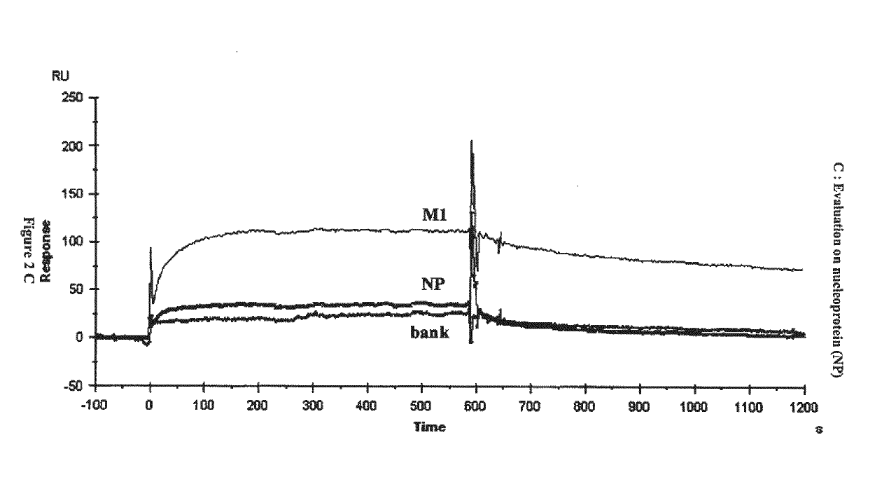

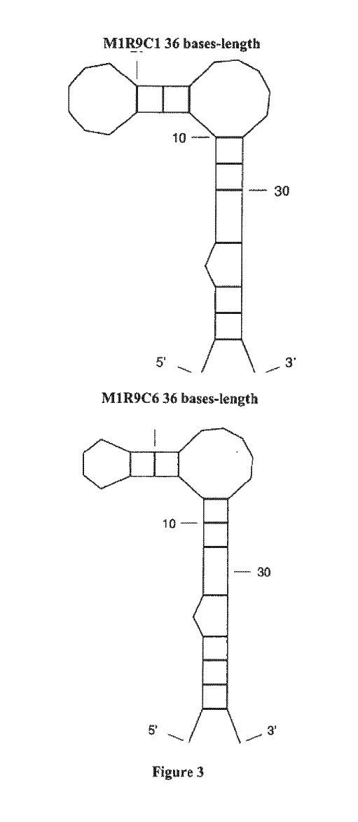

Characterization of shortened aptamer forms—Shortened forms of M1R9C1 and M1R9C6 aptamers were generated by removing the sixteen bases at the 5′ and 3′ ends (SEQ ID NO:8 and 9). These bases corresponded to a part of the invariable domain used for the RT-PCR. All new shortened aptamers showed the same predicted secondary structure (FIG. 3 M1R9C1 36 bases-length and M1R9C6 36 bases-length). Aptamers were organized in a major hairpin which was connected laterally with a small hairpin. Even if the length of the major hairpins differed of two bases, they were characterized by one bulge and one internal loop.

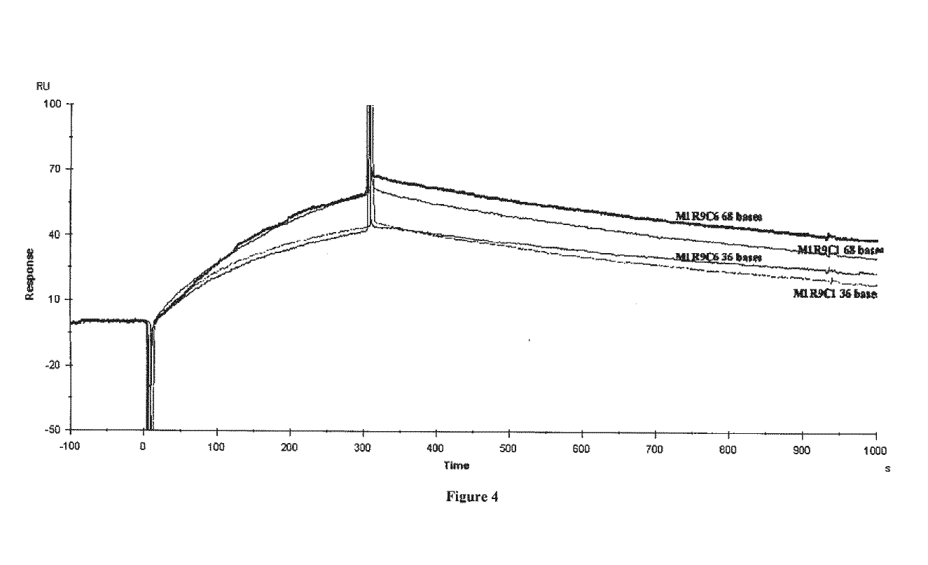

The individual study of shortened aptamer forms showed that M1R9C1 36 bases-length and M1R9C6 36 bases-length kept the binding capacities to matrix protein-1 (FIG. 4). Conversely, the suppression of bulge and internal loop in M1R9C6 36 bases-length caused an almost total loss of the binding capacities. Binding kinetics studies were performed (Table 2; 36 bases-length), using increasing concentrations of aptamers from 0.2 to 8 μM for M1R9C1 36 bases-length and M1R9C6 36 bases-length. The studies revealed a doubling of the equilibrium dissociation constant (KD). M1R9C1 36 bases-length aptamer bound to the matrix protein-1 with association (ka) and dissociation constants (kb) of around 7×10³ (M⁻¹s⁻¹) and 1×10⁻³ (s⁻¹), respectively. The equilibrium dissociation constant (KD) for M1R9C1-M1 complexes was around 2×10⁻⁷ M. In the same way, M1R9C6 36 bases-length aptamer bound to the matrix protein-1 with association (ka) and dissociation constants (kb) of around 7×10³ (M⁻¹s⁻¹) and 9×10⁻⁴ (s⁻²), respectively. The equilibrium dissociation constant (KD) for M1R9C1-M1 complexes was around 2×10⁻⁷ M.

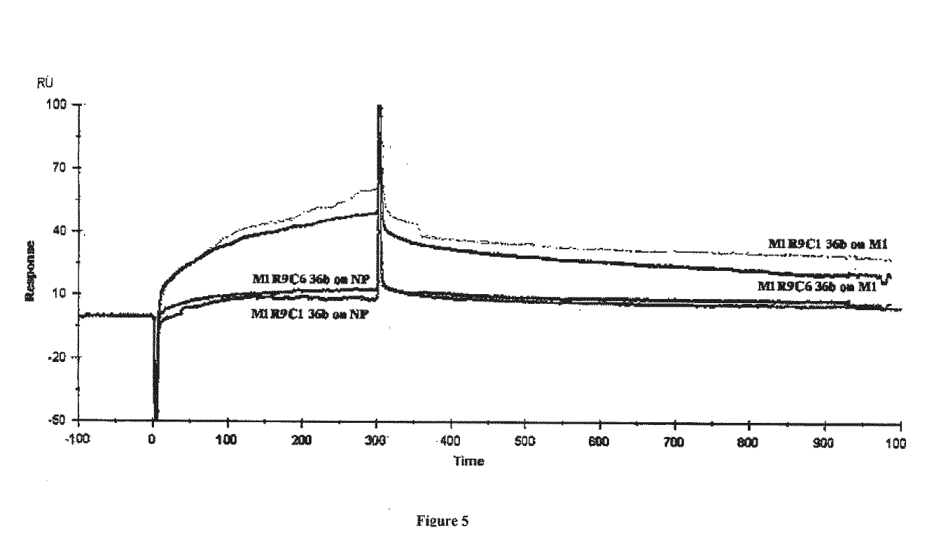

In terms of specificity, the shortened forms of aptamers were tested on nucleoprotein (NP). This viral protein naturally displays a high affinity for ribonucleotides. However, M1R9C1 36 bases-length and M1R9C6 36 bases-length did not complex with NP (FIG. 5).

Chemical modifications of aptamers—In order to confer resistance to nucleases, we introduced modified nucleotides such as 2′-fluoro pyrimidine or DNA. The incorporation of 2′-fluoro pyrimidine induced a dramatic change of the binding capacities, probably due to the non respect of the secondary structure. In contrast, replacement of some ribonucleotides by deoxynucleotides seemed to improve the performance of aptamers. The modifications were located in the lateral loop of molecules. Six bases were replaced in M1R9C1 ARN/DNA, AGAAUC for AGAATC (SEQ ID NO:10). In M1R9C6 ARN/DNA, only four bases were replaced, UGAG for TGAG (SEQ ID NO:11). A kinetic study was performed on DNA modified M1R9C1 36 bases-length (M1R9C1 RNA/DNA). This new form of aptamer bound to the matrix protein-1 with a higher association (ka) of 1×10⁴ (M⁻¹s⁻¹) against 7×10³ (M⁻¹s⁻¹). The equilibrium dissociation constant (KD) was slightly improved, 1×10⁻⁷ M (Table 2).

### EXAMPLE 2

### Aptamer Microarray for the Detection of Influenza Virus

Material & Methods

Capture oligonucléotides—Oligonucleotides were provided at the 40 nmoles synthesis scale (table 1). During the production, a linker was introduced at the 5′ end. The linker was composed of twelve carbons and one amino-group on the 5′ side used for the immobilisation on the slides.

Aptamers detection probes—The following probes were synthesized and coupled to the cy-3 dye at the 5′ side: Cy-3-TGCCGGCCAA (probe C1) (SEQ ID NO:33) and Cy-3-TGCCCGGCCA (probe C6) (SEQ ID NO:34). Probes C1 and C6 were respectively specific of the last 3′ end ten bases of C1 aptamers (i.e. M1R9C1 aptamers) and C6 aptamers (i.e. M1R9C6 aptamers).

Coupling of proteins with fluorescent probes—Matrix protein-1 (100 μg/ml) and total proteins of Vero cells (1 mg/ml) were labelled with cy-5 and cy-3 succinimidyl esters (Amersham Pharmacia, #PA15101 and #PA13101). Briefly, protein solution (23 μl) was mixed to 2 μl of dye (50 μg in DMSO) and 25 μl of labelling buffer (Na₂CO₃, 200 mM, pH 8.3). Proteins were incubated 30 minutes at room temperature in the dark. Labelled proteins were purified by centrifugation (4 min, 400 rpm) on Micro Bio-spin 6 Chromatography column (BioRad, #732-6221). Concentration and dye integration were estimated by UV spectrophotometer at 550 nm and 655 nm. Labelled proteins were stocked at −20° C. in the dark.

Aptamer microarray—Aptamer microarrays were developed on Nexterion Slide E (Schott, distributed by Isogen life science, #1064016). Briefly, capture oligonucleotides were diluted at the 20 μM and 100 μM concentration in the Eurogentec (EGT) spotting buffer. According the manufacturer's recommendations, oligonucleotide solutions were arrayed on the slide surface under controlled atmosphere (40% of humidity) and temperature (22° C.). Slides were incubated in the same conditions overnight and directly stored dry at 4° C.

Quality control—The quality control was performed with 10 bases-length probes DNA oligonucleotides which were specific of the 10 last bases of aptamers. Probes were chemically coupled with the cy-3 dye during synthesis. Cy-3 probes (4 μl) were freshly dissolved in EGT-hybridization buffer (12.5 μl) and DEPC water (7.5 μl). Before hybridization, slides were room temperature equilibrated and conditioned according to manufacturer's instruction. Then, probes were dropped on arrayed areas and recovered with glass cover-slide. After 3 minutes in the dark at 4° C., slides were successively washed for 30 seconds with SSC (2×) SDS (0.1%), SSC (2×) and SSC (0.2×). Slides were dried by centrifugation (1000 rpm, 4 min) and scanned under 360 PMT using GenePix 4100A (Molecular Devices). Quantification was performed using GenePix pro v 5.1 software.

Matrix protein-1 detection—After equilibration at room temperature, slides were washed and blocked with Nexterion buffer according to manufacturer's instructions. Aptamers were folded in SELEX buffer (8 mM Na₂HPO₄ pH 7.5, 140 mM NaCl, 2.5 mM KCl, 1 mM Mg(CH3COO)₂) for 5 minutes at 65° C., 1 minute at 4° C. and 5 minutes at room temperature (22° C.). Slide surfaces were coated in BSA (3%), SELEX buffer during 60 minutes and washed three times (5 min) in PBS, NaCl (0.5M), TWEEN 20 (0.2%). After centrifugation (1000 rpm, 4 min), labelled proteins were dropped on arrayed areas and recovered with cover slide (Invitrogen, #H24723). Slides were placed in hybridization chamber (Corning, #2551) to maintain humidity degree during hybridization (15 minutes, 37° C.; dark). At the end of hybridization, slides were firstly washed in PBS, 0.5 M NaCl and 0.2% TWEEN 20 (three times, 5 minutes); secondly in PBS, 0.1% TWEEN 20; thirdly in PBS and finally in ultrapure water. Before scanning, slides were dried by centrifugation (1000 rpm, 4 min).

Proteins were diluted 50-fold or 250-fold in hybridization buffer composed of PBS pH 7.5; NaCl (0.5 M); TWEEN 20 (0.2%); BSA (3%); MgCl₂ (3 mM); CaCl₂ (0.2 mM) and incubated with tRNA solution for 15 minutes at the final concentration of 0.5 μM or 0.125 μM, respectively. Before use, labelled total proteins of Vero cells were treated with 40 U of RNase inhibitors for 15 min at room temperature (Applied Biosystems, Ambion; SUPERase-In #AM2694).

Results:

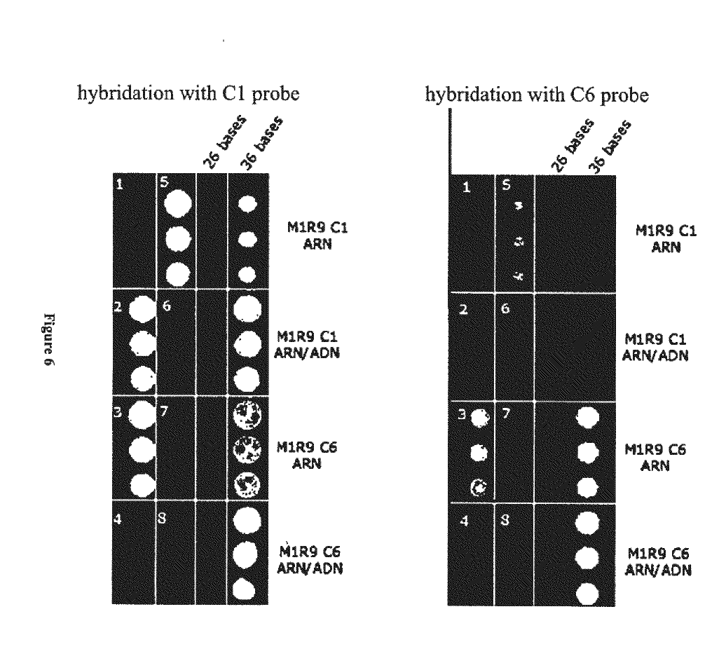

Quality control of the aptamer microarray—Aptamers and control molecules were immobilized on E slide surface using 5′-amino groups. Before the use of the microarray in the detection of the matrix protein-1, slides underwent several treatments as blocking and folding of aptamers. These could damage aptamers or their binding to the surface. The quality control step consisted of hybridization with cy-3 probe complementary of the last ten 3′ end bases of the aptamer and specific of aptamers C1 or aptamers C6. FIG. 6 showed respectively the typical results for the probe C1 (left) and C6 (right). In the case of probe C1, a significant fluorescence signal was observed for the positive controls in positions 2 and 3 and for the different forms of 36 bases-length aptamers. In the same way, the negative controls corresponding to the 26 bases-length aptamers were not visible. The positive signal observed in area 5 was due to the high identity between M1R9C1 DNA and aptamers C1.

Similar results were observed with the probe C6. However, probe C6 appeared to be more specific than probe C1 since the fluorescence signal was higher for molecules derived from C6 than molecules derived from C1. Note that nonspecific signals were not observed in the areas 1 and 4. These observations were confirmed by the quantification of the fluorescence signals (Table 3). In the case of probe C1, the maximal values were obtained for the molecules derived from the aptamer C1. The highest detection was observed for the oligo C1 (13.148), and progressively less intense signal was retrieved for the M1R9C1 DNA, M1R9C1 RNA/DNA and M1R9C1, respectively. In the second place, the molecule derived from aptamer C6 were classified with a maximal signal for oligo C6 and a minimal signal for

M1R9C6. The negative controls showed a very low signal, closed to the detection limit. Note that the higher value obtained for oligo C6 in comparison to M1R9C1 was probably due to the difference of nature between these molecules. The deoxyribonucleotidic nature of oligo C6 was more favorable to hybridization with DNA probe in comparison with M1R9C1 that was strictly composed in ribonucleotides. In the case of utilization of the C6 probe, similar data were observed. However, C6 probe displayed a better specificity and allows to discriminate between the molecules that derived from aptamers C1 or C6. The maximal values were obtained for M1R9C6 RNA/DNA, M1R9C6 and oligo C6. All of them were higher than 10,000 while the molecules derived from aptamer C1 showed a fluorescence signal around 1,000 with the exception of M1R9C1 DNA. The negative controls showed a fluorescence signal close to the detection limit.

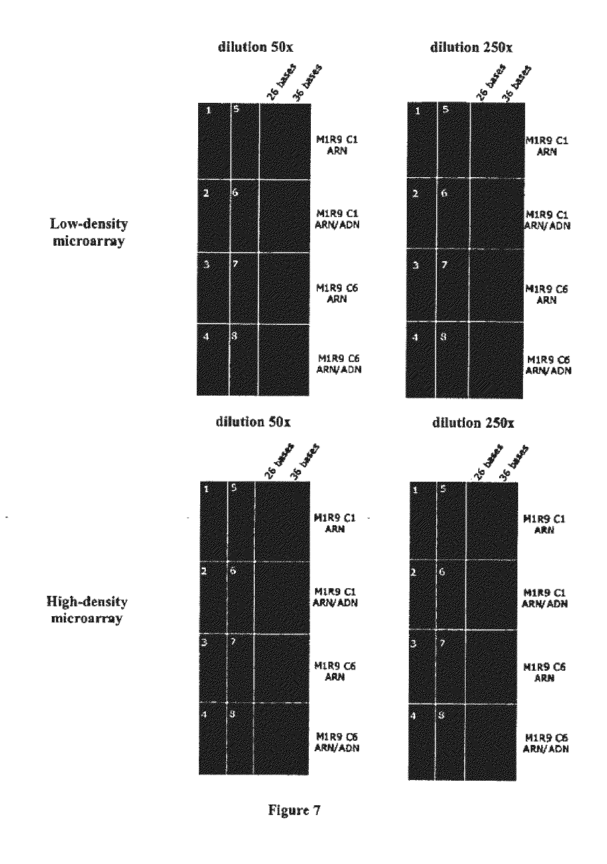

Functionality of the aptamer microarray to detect the matrix protein-1—The first step of the validation of aptamer microarray for the detection of influenza virus consisted of the study of the ability to detect the purified target. For this, a stock solution of cy-5 labeled matrix protein-1 was diluted 50 fold and 250 fold in hybridization buffer. After saturation of nonspecific sites and folding of aptamers, samples were dropped on arrayed area for 15 minutes at 37° C. in the dark. After washing, slides were scanned under 650 PMT using GenePix 4100A apparatus. Two kind of slides were used, one arrayed with oligonucleotide solution at 20 μM (low-density microarray) and another arrayed with oligonucleotide solution at 100 μM (high-density microarray). For all tests, we observed the same patterns of detection (FIG. 7). Only the aptamers of 36 bases-length were able to form complexes with cy-5 target and no background or nonspecific bindings were observed. Low quantities of target protein could be detected by this system since positive signal was observed for a 250-fold dilution (10 ng). However, the four aptamers did not show the same capacities to recognize the matrix protein-1. Indeed, spots were more visible in the areas of aptamers M1R9C6 RNA/DNA and M1R9C6 than in the areas arrayed with aptamers derived from C1. The quantification of fluorescence signal by GenePix v 5.1 software confirmed these results. In our assays with different dilutions and low-density or high-density microarrays, the M1R9C6 and M1R9C6 RNA/DNA aptamers were the most effective for the detection of matrix protein-1 (Table 4). Coming later in decreasing order: M1R9C1 RNA/DNA and M1R9C1. Negative controls showed a very low signal level which was below the detection limit. Use of high density microarray increased signal at least twice, from 7.368 to 17.165 for M1R9C6 aptamer, whereas, the signal for negative controls did not increase and stayed at the detection limit. Note that there was a direct relationship between the signal level and the amount of target protein. Indeed, the 5-fold dilution of the sample resulted in a decrease in the signal level of an identical coefficient.

In conclusion, our aptamer microarray allows unambiguous detection of the matrix protein-1, even in low quantity (0.33×10⁻¹² moles).

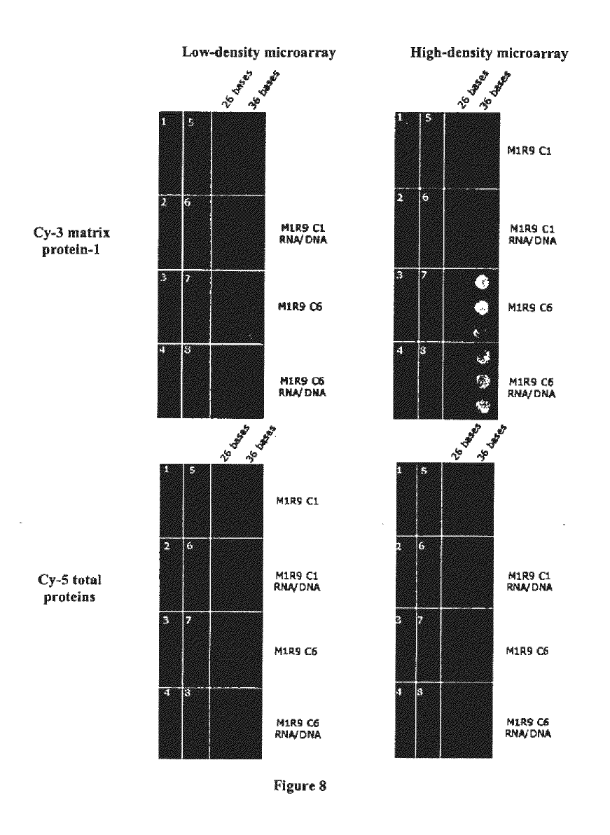

Detection of matrix protein-1 in a complex medium—For this work, the matrix protein-1 was mixed with total proteins from Vero cells. After freeze-thaw extraction, total proteins were labeled by the cy-5 dye. Note that matrix protein-1 was labeled in the same conditions with the cy-3 dye. Before mixing, total proteic extract from Vero cells was treated with RNase inhibitors during 10 min. The two preparations of proteins were then mixed (ratio 1:1) and diluted in the hybridization buffer. Hybridization was performed in the dark at 37° C. for 15 min. In order to detect a nonspecific binding of cy-5 labeled cellular proteins, the detection level was increased at 750 PMT. In these conditions, the patterns of detection shown in FIG. 8 were obtained. Firstly, specific complexes were formed between matrix protein-1 and aptamers. As noted earlier, M1R9C6 and M1R9C6 RNA/DNA aptamers seemed more efficient than M1R9C1 and M1R9C1 RNA/DNA. A very weak nonspecific signal was observed on slides. Secondly, no complex was observed between aptamers and cy-5 cellular proteins. These observations were in accordance with the high specificity of our aptamers shown by SPR. The majority of the Cy-5 signal was located outside of arrayed areas.

The quantification confirmed the high ability of M1R9C6 RNA/DNA and M1R9C6 to detect the matrix protein-1 in complex medium (Table 5). The signal reached 3780 for M1R9C6 RNA/DNA on low-density microarray and could be increased by use of a high-density microarray to reach 5222. In contrast, the fluorescence signals for aptamers derived from C1 stayed medium and did not increase with the utilization of a high-density microarray. The fluorescence signal for negative control was significantly lower than that retrieved from aptamers. If we consider only the most efficient aptamers, the signal of negative controls did not exceed more than 15.2% of the positive signal (M1R9C6 RNA/DNA, table 5 A). Our microarrays displayed a high degree of specificity for matrix protein-1 of Influenza virus since fluorescence signal for cy-5 total protein was under detection limit.

TABLE 1
Sequences of molecules immobilized on the
microarray

| aptamers | |
|---|---|
| 36 bases- | |
| length | |
| M1R9C1 | UGCCUGACCACUCAGAAUCGAGCGCAUUGGCCGGCA |
| | (SEQ ID NO: 8) |
| M1R9C1 | UGCCUGACCACUCAGAATCGAGCGCAUUGGCCGGCA |
| RNA/DNA | (SEQ ID NO: 10) |
| M1R9C6 | UGCCCUGACCAUCCUGAGGGACGCAUUGGCCGGGCA |
| | (SEQ ID NO: 9) |
| M1R9C6 | UGCCCUGACCAUCCTGAGGGACGCAUUGGCCGGGCA |
| RNA/DNA | (SEQ ID NO: 10) |
| aptamers | |
| 26 bases- | |
| length | |
| M1R9C1 | UGCCUGACCACUCAGAAUCGAGCGCA |
| | (SEQ ID NO: 35) |
| M1R9C1 | UGCCUGACCACUCAGAATCGAGCGCA |
| RNA/DNA | (SEQ ID NO: 36) |
| M1R9C6 | UGCCCUGACCAUCCUGAGGGACGCAU |
| | (SEQ ID NO: 37) |
| M1R9C6 | UGCCCUGACCAUCCTGAGGGACGCAU |
| RNA/DNA | (SEQ ID NO: 38) |
| control | |
| oligo C1 | TTGGCCGGCA (SEQ ID NO: 33) |
| oligo C6 | TGGCCGGGCA ′SEQ ID NO: 34) |
| M1R9C1 | TGCCTGACCACTCAGAATCGAGCGCATTGGCCGGCA |
| DNA | (SEQ ID NO: 39) |
| anti- | GGCGAACCGAUGGAAUUUUUGGACGCUCGCC (SEQ ID |
| VEGF | NO: 40) |
| anti- | CAGGCTACGGCACGTAGAGCATCACCATGATCCT (SEQ |
| PDGF | ID NO: 41) |
| MIR9C6 | UGCCCUGGCCAUCCUGAGGGACGCAUUGGCCAGGGCA |
| bulge | (SEQ ID NO: 42) |
| less | |

Oligonucleotides were synthesized at the 40 nmoles scale. During the synthesis a linker of 12 carbons was introduce on the 5′ end. The linker was chemical modified to form an amino group. The underline sequences were in DNA.

TABLE 2
Kinetic components of aptamers

| | Ka (1/Ms) | KD (1/s) | KD (M) | Chi2 |
|---|---|---|---|---|
| 68 bases-length | | | | |
| M1R9C1 | 81 10³ | 2 10⁻³ | 4 10⁻⁷ | 4 |
| M1R9C6 | 4 10³ | 10⁻³ | 3 10−⁷ | 2 |
| 36 bases-length | | | | |
| M1R9C1 | 7 10³ | 10⁻³ | 2 10⁻⁷ | 0.2 |
| M1R9C6 | 7 10³ | 9 10⁻⁴ | 2 10⁻⁷ | 0.1 |
| M1R9C1 | 10⁴ | 1.5 10⁻³ | 1 10⁻⁷ | 3 |

RNA/DNA

Binding kinetic studies were performed by successive injections of aptamers at increasing concentrations from 0.1 μM to 10 μM (22° C., 20 μ/min).

TABLE 3
Quantification of the quality control
Low-density microarray

| probe C1 | | | | probe C6 | | | |
|---|---|---|---|---|---|---|---|
| | 36 bases | 26 bases | control | | 36 bases | 26 bases | control |
| M1R9C1 | 6128 | 118 | | M1R9C1 | 868 | 13 | |
| M1R9C1 RNA/DNA | 10233 | 15 | | M1R9C1 RNA/DNA | 1027 | 69 | |
| M1R9C6 | 3662 | 8 | | M1R9C6 | 11823 | 3 | |
| M1R9C6 RNA/DNA | 5268 | 65 | | M1R9C6 RNA/DNA | 15236 | 61 | |
| spotting buffer | | | 13 | spotting buffer | | | 1 |
| oligo c1 | | | 13148 | oligo c1 | | | 1791 |
| oligo c6 | | | 8426 | oligo c6 | | | 10812 |
| tRNA | | | 23 | tRNA | | | 9 |
| M1R9C1 ADN | | | 10964 | M1R9C1 ADN | | | 4024 |
| anti-VEGF | | | 19 | anti-VEGF | | | 25 |
| anti-PDGF | | | 671 | anti-PDGF | | | 86 |
| MIR9C6 bulgeless | | | 117 | MIR9C6 bulgeless | | | 346 |

Probes were applied on aptamer microarray for 30 minutes at 4° C. Quantification of fluorescence level was performed on GenePix 4100A instrument using GenePix Pro 5.1 software under 360 PMT. Values came from the average of the three spots after the subtraction of the background.

TABLE 4
Quantification of the level of matrix protein-1 detection

| Low-density microarray | | | | High-density microarray | | | |
|---|---|---|---|---|---|---|---|
| | 36 bases | 26 bases | control | | 36 bases | 26 bases | control |

A
Dilution 50x

| | | | | | | | |
|---|---|---|---|---|---|---|---|
| M1R9C1 | 1914 | −328 | | M1R9C1 | 4376 | 1139 | |
| M1R9C1 RNA/DNA | 3180 | −834 | | M1R9C1 RNA/DNA | 7553 | −791 | |
| M1R9C6 | 7368 | −456 | | M1R9C6 | 17165 | 188 | |
| M1R9C6 RNA/DNA | 6979 | −141 | | M1R9C6 RNA/DNA | 15930 | −1063 | |
| spotting buffer | | | −4 | spotting buffer | | | −116 |
| oligo c1 | | | 1175 | oligo c1 | | | 443 |
| oligo c6 | | | 619 | oligo c6 | | | 506 |
| tRNA | | | −55 | tRNA | | | 2946 |
| M1R9C1 ADN | | | 1202 | M1R9C1 ADN | | | −2050 |
| anti-VEGF | | | 1112 | anti-VEGF | | | 340 |
| anti-PDGF | | | −796 | anti-PDGF | | | −1396 |
| MIR9C6 bulgeless | | | −264 | MIR9C6 bulgeless | | | −942 |

B
Dilution 250x

| | | | | | | | |
|---|---|---|---|---|---|---|---|
| M1R9C1 | 464 | −30 | | M1R9C1 | 394 | −58 | |
| M1R9C1 RNA/DNA | 859 | −43 | | M1R9C1 RNA/DNA | 834 | −100 | |
| M1R9C6 | 1715 | −4 | | M1R9C6 | 2609 | −10 | |
| M1R9C6 RNA/DNA | 1727 | −10 | | M1R9C6 RNA/DNA | 2259 | −88 | |
| spotting buffer | | | 20 | spotting buffer | | | −20 |
| oligo c1 | | | 31 | oligo c1 | | | −24 |
| oligo c6 | | | −16 | oligo c6 | | | −38 |
| tRNA | | | −40 | tRNA | | | 2 |
| M1R9C1 ADN | | | 6 | M1R9C1 ADN | | | −52 |
| anti-VEGF | | | −8 | anti-VEGF | | | −17 |
| anti-PDGF | | | −106 | anti-PDGF | | | −83 |
| MIR9C6 bulgeless | | | −80 | MIR9C6 bulgeless | | | −135 |

Target protein was diluted 50× (A) or 250× (B) in the hybridization buffer and applied on low-density and high density microarray for 15 minutes at 37° C. Quantification of fluorescence level was performed on GenePix 4100A instrument using GenePix Pro 5.1 software under 650 PMT. Values came from the average of the three spots after the subtraction of the background.

TABLE 5
Quantification of matrix protein-1 detection in a complex medium

| Fluorescence signal for cy-3 labeled matrix protein-1 | | | | Fluorescence signal for cy-5 labeled total protein | | | |
|---|---|---|---|---|---|---|---|
| | 36 bases | 26 bases | control | | 36 bases | 26 bases | control |

A
Low-density microarray

| | | | | | | | |
|---|---|---|---|---|---|---|---|
| M1R9C1 | 1344 | 409 | | M1R9C1 | −989 | −775 | |
| M1R9C1 RNA/DNA | 1064 | 628 | | M1R9C1 RNA/DNA | 83 | 219 | |
| M1R9C6 | 2860 | −22 | | M1R9C6 | −137 | −497 | |
| M1R9C6 RNA/DNA | 3780 | 577 | | M1R9C6 RNA/DNA | −842 | −605 | |
| spotting buffer | | | 49 | spotting buffer | | | 239 |
| Oligo C1 | | | 351 | oligo C1 | | | −121 |
| Oligo C6 | | | 99 | oligo C6 | | | −98 |
| TRNA | | | 85 | tRNA | | | 65 |
| M1R9C1 DNA | | | −364 | M1R9C1 DNA | | | −18 |
| Anti-VEGF | | | 619 | antiVEGF | | | −779 |
| anti-PDGF | | | −899 | anti-PDGF | | | −1035 |
| MIR9C6 bulgeless | | | 136 | MIR9C6 bulgeless | | | −664 |

B
High-density microarray

| | | | | | | | |
|---|---|---|---|---|---|---|---|
| M1R9C1 | 1513 | 245 | | M1R9C1 | −893 | −1190 | |
| M1R9C1 RNA/DNA | 1527 | 731 | | M1R9C1 RNA/DNA | −747 | −891 | |
| M1R9C6 | 5614 | 684 | | M1R9C6 | −608 | −863 | |
| M1R9C6 RNA/DNA | 5222 | 72 | | M1R9C6 RNA/DNA | −1411 | −1381 | |
| spotting buffer | | | 732 | spotting buffer | | | 1081 |
| oligo C1 | | | 408 | oligo C1 | | | −985 |
| oligo C6 | | | 595 | oligo C6 | | | −717 |
| TRNA | | | 489 | tRNA | | | −133 |
| M1R9C1 DNA | | | −596 | M1R9C1 DNA | | | −78 |
| anti-VEGF | | | 570 | anti-VEGF | | | −965 |
| anti-PDGF | | | −1018 | anti-PDGF | | | −946 |
| MIR9C6 bulgeless | | | −737 | MIR9C6 bulgeless | | | −1770 |

Cy-3 target protein was mixed to cy-5 total protein of Vero cells at a ration 1:1. The mixture was diluted 50× in the hybridization buffer and applied on low-density (A) and high-density (B) microarrays for 15 minutes at 37° C. Slides were scanned successively at 550 nm and 655 nm on GenePix 4100A instrument under 750 PMT. Data were analyzed with GenePix Pro 5.1 software. Values came from the average of the three spots after the subtraction of the background.

### REFERENCES

Throughout this application, various references describe the state of the art to which this invention pertains. The disclosures of these references are hereby incorporated by reference into the present disclosure.

1. A nucleic acid that binds specifically to matrix protein-1 of type A influenza viruses characterized in that said nucleic acid comprises the following nucleotide sequence:
5′-N1-NS1-U-N3-A-NS3-NS5-NS7-NS6-CGCAU-NS4-C-N4-
NS2-N2-3′
wherein:
N1 consists of a nucleotide
NS1 and NS2 consist of polynucleotides having 3 or 4 nucleotides in length, and NS1 and NS2 have complementary sequences;
N3 and N4 consists of a nucleotide, and N4 is complementary to N3;
NS3 and NS4 consist of polynucleotides having 3 nucleotides in length, and NS3 and NS4 have complementary sequences
NS5 and NS6 consist of polynucleotides having 3 nucleotides in length, and NS5 and NS6 have complementary sequences;
NS7 consists of a polynucleotide selected from the group consisting of AGAAUC (SEQ ID NO:12), UGAG (SEQ ID NO:13), UAUUCC (SEQ ID NO:14), AGAU (SEQ ID NO:15), AGAATC (SEQ ID NO:16) or TGAG (SEQ ID NO:17), and
N2 consists of a nucleotide that is complementary or not complementary to nucleotide N1.

2. The nucleic acid according to claim 1 wherein N1 is U and N2 is A.

3. The nucleic acid according to claim 1 wherein NS1 is GCC (SEQ ID NO:18) and NS2 is GGC (SEQ ID NO:19).

4. The nucleic acid according to claim 1 wherein NS1 is GCCC (SEQ ID NO:20) and NS2 is GGGC (SEQ ID NO:21).

5. The nucleic acid according to claim 1 wherein N3 is G and N4 is C.

6. The nucleic acid according to claim 1 wherein NS3 is CCA (SEQ ID NO:22) and NS4 is UGG (SEQ ID NO:23).

7. The nucleic acid according to claim 1 wherein NS5 is CUC (SEQ ID NO:24) and NS6 is GAG (SEQ ID NO:25).

8. The nucleic acid according to claim 1 wherein NS5 is UCC (SEQ ID NO:26) and NS6 is GGA (SEQ ID NO:27).

9. The nucleic acid according to claim 1 wherein NS5 is CCU (SEQ ID NO:28) and NS6 is AGG (SEQ ID NO:29).

10. The nucleic acid according to claim 1 which comprises a nucleic acid sequence selected from the group consisting of SEQ ID NO:4 (M1R9C1), SEQ ID NO:5 (M1R9C6), SEQ ID NO:8 (M1R9C1 36 bases length), SEQ ID NO:9 (M1R9C6 36 bases length), SEQ ID NO:10 (M1R9C1 RNA/DNA 36 bases length) and SEQ ID NO:11 (M1R9C6 RNA/DNA 36 bases length).

11-13. (canceled)

14. A microarray comprising a solid support which carries at least one nucleic acid according to claim 1.

15. A kit comprising at least one nucleic acid according to claim 1.

16. The nucleic acid according to claim 1 which consists of a nucleic acid sequence selected from the group consisting of SEQ ID NO:4 (M1R9C1), SEQ ID NO:5 (M1R9C6), SEQ ID NO:8 (M1R9C1 36 bases length), SEQ ID NO:9 (MIR9C6 36 bases length), SEQ ID NO:10 (M1R9C1 RNA/DNA 36 bases length) and SEQ ID NO:11 (M1R9C6 RNA/DNA 36 bases length).

17. A method of detecting and/or quantifying matrix protein-1 in a sample of interest, comprising the steps of
a) providing a sample to be tested;
b) bringing into contact said sample and one or more nucleic acids according to claim 1;
c) detecting complexes formed between matrix protein-1 proteins in said sample and said one or more nucleic acids; and, optionally
d) quantifying said complexes.

18. The method according to claim 17, wherein said sample of interest is selected from the group consisting of laboratory cultures, nasopharyngeal washes, expectorate, respiratory tract swabs, throat swabs, tracheal aspirates, bronchoalveolar lavage, mucus and saliva.

19. A method of detecting and/or quantifying a type A Influenza virus in a sample of interest, comprising the step of
a) providing a sample to be tested;
b) bringing into contact said sample and one or more nucleic acids according to claim 1;
c) detecting complexes formed between matrix protein-1 proteins in said sample and the one or more nucleic acids, wherein the presence of such complexes is indicative of the presence of a type A influenza virus in said sample; and, optionally
d) quantifying said complexes.

20. The method according to claim 19, wherein said sample of interest is selected from the group consisting of laboratory cultures, nasopharyngeal washes, expectorate, respiratory tract swabs, throat swabs, tracheal aspirates, bronchoalveolar lavage, mucus and saliva.
